(19) United States
(12) Patent Application Publication
(10) Pub. No.: US 2005/0244883 A1
(43) Pub. Date: Nov. 3, 2005

(54) Method and computer software product for genomic alignment and assessment of the transcriptome

(75) Inventors: Alan J. Williams, Kensington, CA (US); Simon Cawley, Oakland, CA (US); Raymond Wheeler, Berkeley, CA (US); Brant Wong, Walnut Creek, CA (US); David Kulp, Albany, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/168,777

(22) Filed: Jun. 27, 2005

Related U.S. Application Data
(63) Continuation of application No. 11/090,674, filed on Mar. 24, 2005.
Application No. 11/090,674 is a continuation of application No. 10/028,884, filed on Dec. 21, 2001, now abandoned.

(51) Int. Cl. C12Q 1/68; G06F 19/00; G01N 33/48; G01N 33/50

(52) U.S. Cl. 435/6; 702/20

(57) ABSTRACT

In one embodiment of the invention, computerized methods are provided for analyzing transcript sequence clusters by aligning the transcripts with genomic sequences to determine whether a cluster needs to be sub-clustered and whether clusters should be merged. In addition, in some embodiments, transcript sequences are trimmed according to their alignment with their corresponding genomic sequences. The modified clusters and trimmed sequences may be used for nucleic acid probe array design.

### RELATED APPLICATIONS

This application is related to U.S. patent application Ser. No. 09/721,042, filed on Nov. 21, 2000, entitled “Methods and Computer Software Products for Predicting Nucleic Acid Hybridization Affinity”; U.S. patent application Ser. No. 09/718,295, filed on Nov., 21, 2000, entitled “Methods and Computer Software Products for Selecting Nucleic Acid Probes”; U.S. patent application Ser. No. 09/745,965, filed on Dec. 21, 2000, entitled “Methods For Selecting Nucleic Acid Probes”; nd U.S. patent application Ser. No. ______, attorney Docket No. 3439, filed on Dec. 21, 2001, and U.S. patent application Ser. No. ______, attorney docket number 3440, filed on Dec. 21, 2001. All the cited applications are incorporated herein by reference in their entireties for all purposes.

### BACKGROUND OF THE INVENTION

This invention is related to bioinformatics and biological data analysis. Specifically, this invention provides methods, computer software products and systems for determining the orientation of biological sequence clusters. In preferred embodiments, the methods, computer software products and systems are used for designing nucleic acid probe arrays.

Transcriptome refers to the complete collection of RNAs that are transcribed from a genome. Expressed sequence tags (ESTs) offer a rapid and relatively inexpensive way to chacracterize the transcriptome (Vasmatis et al., 1998, Discovery of three genes specifically expressed in human prostate by expressed sequence tag database analysis, Proc. Natl. Acad. Sci. USA 95(1):300-304; Adams et al., 1991, Complementary DNA sequencing: Expressed Sequence Tags and Human Genome Project. Science 252:1651-1656, both incorporated herein by reference for all purposes). cDNA sequence clusters including EST and mRNA sequences as well as hypothetical mRNA sequences predicted by computation methods offer a view of the transcriptome and have been used for nucleic acid probe array design (U.S. Pat. No. 6,188,783, incorporated herein by reference) and for the discovery of new human genes, mapping of the human genome, and identification of coding regions in genomic sequences (Adams, et al., 1991, Science 252:1651-1656).

Because a large amount of EST data are produced using single-read sequencing in a high throughput setting, they tend to be error-prone and as a result, the understanding of the transcriptome gained from the EST data can be unreliable.

### SUMMARY OF THE INVENTION

In one aspect of the invention, transcript sequences are aligned to their corresponding genome based upon sequence alignment. In some embodiments, the alignment information is used to infer low quality sequence regions in cDNA sequences (including but not limited to EST sequences) based on genomic alignments, to identify chimeric clusters of sequences (where those clusters may have been generated by a transcriptome based clustering method such as is done for UniGene) and appropriately subcluster based on the genomic alignments of the individual sequences and to merge clusters based on genomic alignments.

In preferred embodiments, methods are provided for analyzing the sequence quality of cDNA sequences such as ESTs and plurality of sequences within a cluster to detect and assess chimeric clusters, and clusters that should be merged. The methods include aligning the transcript sequences with genomic sequences. Given these alignments, cDNA sequence quality as well as whether the clusters need to be modified can be determined according to the alignments. For cDNA sequence quality, the aligned portion of the cDNA sequence can be considered high quality while the unaligned portion can be considered low quality. For clusters, the step of determining may include classifying a cluster as a chimeric cluster if the cluster is aligned to two or more separate locations in the genomic sequence. In particularly preferred embodiments, a chimeric cluster contains sequences which align to different positions in the genomic sequence. “Different positions” could refer to different genomic regions entirely or to the lack of overlapping exonic regions. Strand information could be used or ignored in this asssessment process. The methods may also include additional steps of subclustering the chimeric clusters.

In additional embodiments, the step of determining includes detecting clusters which overlap or are within a certain distance from each other, for example, within 1000 bases, in genomic space and optionally merging the clusters. This analysis could be performed with the member sequences of each cluster and/or the consensus sequence for a cluster. In most cases the strandedness of the sequences would be taken into account, but could be ignored.

In some instances, methods for triming a transcript sequence and detecting chimeric sequences are provided. The methods include aligning the transcript sequence to its correponding genomic sequence or sequences. Poorly aligned regions or regions which do not align can be treated as low quality while the aligned portion can be treated as high quality. Furthermore, the low quality region can be removed, creating a “trimned” version of the sequence containing only the high quality region(s). When mutually exclusive portions of the transcript sequence align to distant portions of the genome or align in a non-linear fashion, the transcript can be considered chimeric. Furthermore, two or more new sequences can be created based on the transcript alignments to the genome. “Distant” can refer to regions on different chromosomes, different strands of the same chromosome or regions sufficiently appart on the same chromosome and strand such that the distance is not likely to be an intron. Non-linear alignments occur when the order of the aligned regions in the genomic sequence is different than the order of the regions within the transcript. The quality of the genomic sequence can optionally be taken into account such that these annotations and actions occur only for genomic sequence of a specific quality (such as finished).

In another aspect of the invention, methods for designing a nucleic acid probe array are provided. The methods include aligning a plurality of transcript sequence clusters to their corresponding genomic sequence; modifying the clusters according to their aligning to the genomic sequence to obtain modified clusters; and selecting probes targeting the modified clusters. The step of modifying may include subclustering chimeric clusters. In preferred embodiments, a cluster is classified as a chimeric cluster if the cluster is aligned to two or more separate locations in the genomic sequence. The step of modifying comprises merging the clusters with consensus which overlap in genomic space.

In some preferred embodiments, the methods include merging the clusters with consensus within 1000 bases and on the same strand or clusters which overlap in the genome space.

Methods are also provided for designing nucleic acid probe arrays using trimmed transcripts for probe selection. The methods include aligning a transcript sequence to its corresponding genomic sequence; triming a side of the transcript sequence to obtain a trimmed transcript sequence if the side of the transcript sequence is poorly align with the genomic sequence; and selecting probes targeting the trimmed transcript sequence or clusters including the trimmed transcript sequence.

In another aspect of the invention, systems and computer software are provided for performing the methods of the invention. The systems include a processor, and a memory coupled with the processor, the memory storing a plurality of machine instructions that cause the processor to perform logical steps of the methods of the invention. The computer software products of the invention include a computer readable medium having computer-executable instructions for performing the method of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary computer system suitable for executing some embodiments of the software of the invention.

FIG. 2 is a schematic showing the architecture of the exemplary computer system of FIG. 1.

FIG. 3 shows an exemplary computer network system suitable for executing some embodiments of the software of the invention.

FIG. 4 shows an exemplary process for using genomic aligning, subclustering and cluster merging.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention will employ, unless otherwise indicated, conventional techniques of bioinformatics, computer sciences, immunology, biochemistry, chemistry, molecular biology, microbiology, cell biology, genomics and recombinant DNA, which are within the skill of the art. See, e.g., Setubal and Meidanis, et al., 1997, Introduction to Computational Molecular Biology, PWS Publishing Company, Boston; Human Genome Mapping Project Resource Centre (Cambridge), 1998, Guide to Human Genome Computing, 2nd Edition, Martin J. Biship (Editor), Acadernic Press, San Diego; Salzberg, Searles, Kasif, (Editors), 1998, Computational Methods in Molecular Biology, Elsevier, Amsterdan; Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987))

System for Sequence Annotation and for Nucleic Acid Probe Array Design

In aspects of the invention, methods, computer software and systems for determining the orientation of EST sequence clusters and for probe array design are provided. One of skill in the art would appreciate that many computer systems are suitable for carrying out the methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

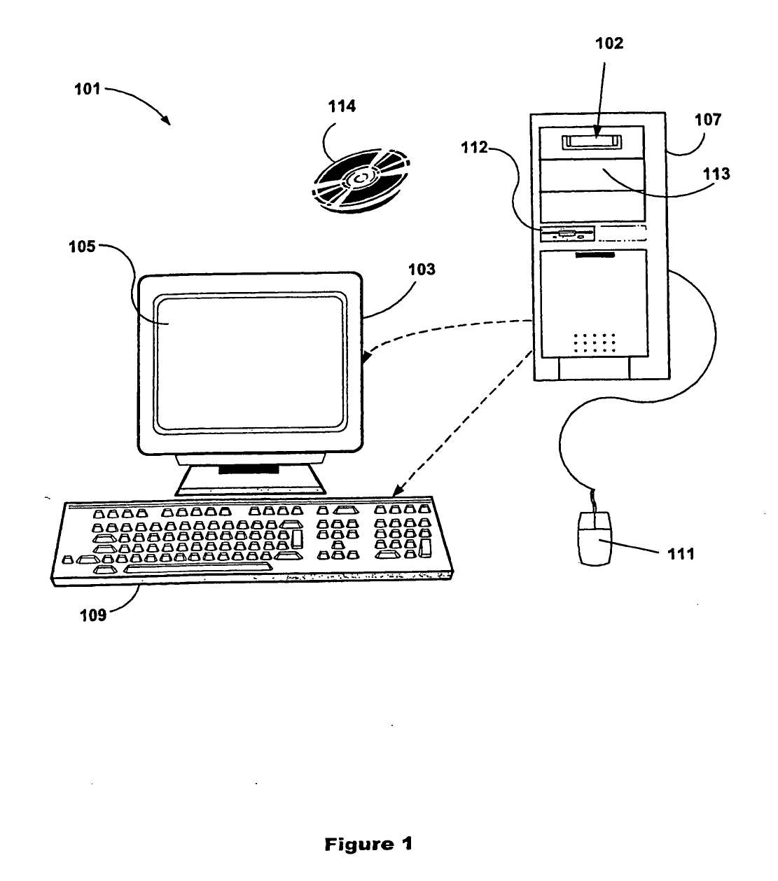

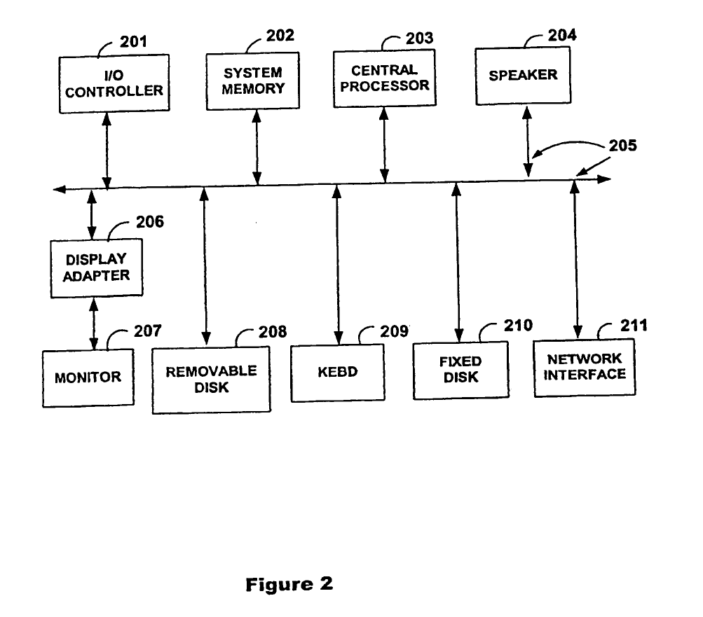

FIG. 1 illustrates an example of a computer system that may be used to execute the software of an embodiment of the invention. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

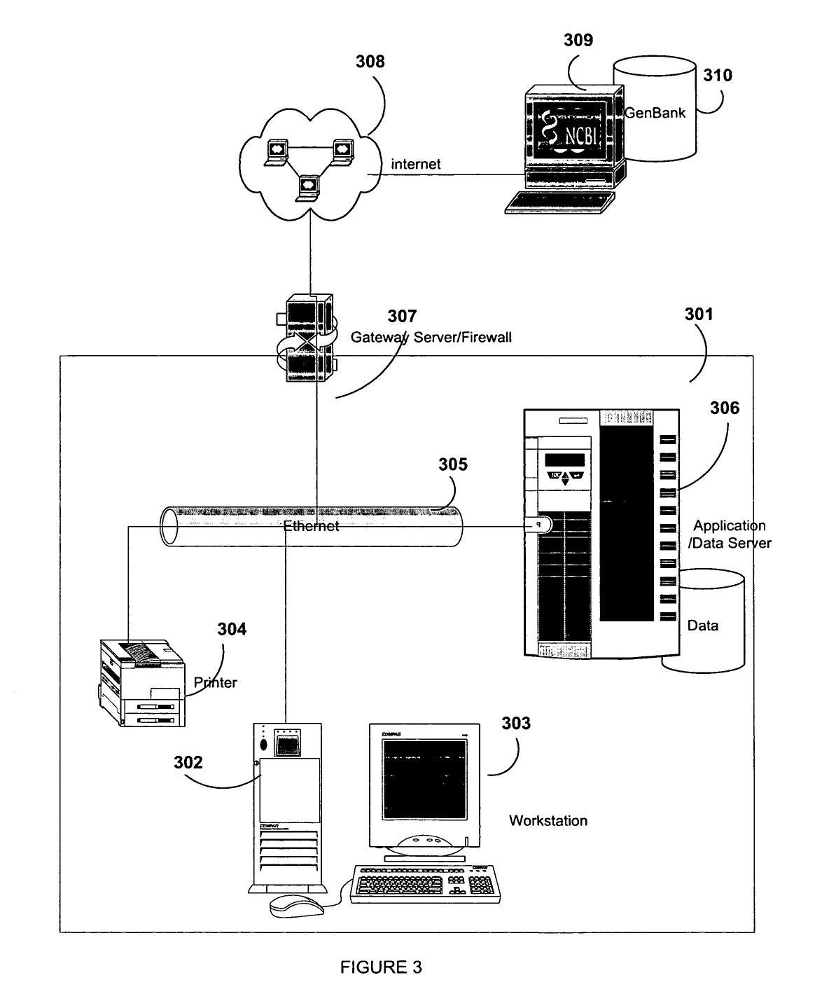

FIG. 3 shows an exemplary computer network that is suitable for executing the computer software of the invention. A computer workstation 302 is connected with the application/data server(s) through a local area network (LAN) 301, such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In preferred embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (“www.ncbi.nlm.nih.gov”).

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++(such as Visual C/C++), C#, Java, Basic (such as Visual Basic), SQL, Fortran, SAS and Perl.

Nucleic Acid Probe Arrays

The methods, computer software and systems of the invention are particularly useful for designing high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138 and 6,040,193, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques. See also, Fodor, et al., Science, 251, 767-77 (1991). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarrays can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hum. Mut. 7:244-255, both incorporated by reference in their entireties), the human immunodeficiency virus (HIV-1) reverse transcriptase and protease genes (U.S. Pat. No. 5,862,242 and Kozal et al., 1996, Extensive polymorphisms observed in HIV-1 clade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic Acid Probe Array Design Process

In some embodiments, a nucleic acid probe array design process involves selecting the target sequences and selecting probes. For example, if the probe array is designed to detect the expression of genes at the transcript level. The target sequences are typically transcript sequences. Selection of the target sequence may involve the characterization of the target sequence based upon available information. For example, expressed sequence tags information needs to be assembled and annotated.

After target sequences are identified, probes for detecting the target sequences can be selected. The probe sequences and layout information are then translated to photolithographic masks, commands for controlling ink-jet directed synthesis, or soft lithographic synthesis process.

EST and Sequence Clusters

Nucleic acid probe array design, particular for arrays detecting gene expression, often involves the analysis of an “EST”, or “Expressed Sequence Tag”. EST as used herein, refers to a fragment of a cDNA clone that has been at least partially sequenced. For a detailed discussion of the process of producing ESTs, see.e.g., Baldo et al., 1996, Normalization and Substraction: Two Approaches to Facilitate Gene Discovery. Genome Research 6:791-806, which is incorporated herein by reference for all purposes.

With the easy access to technology to generate ESTs, tens of thousands of ESTs are sequenced. The high volume and high throughput nature of the EST data often results in high error rates (Aaronson et al., 1996, et al., Toward the Development of a Gene Index to the Human Genome: An Assessment of the Nature of High Throughput EST sequence Data. Genome Research 6:829-845, incorporated herein by reference for all purposes). Typically, a single read is generated for each EST. Errors typically include wrong clone orientation, associated clone ID chimeras, missing 3′ and 5′ reads.

A common way to assemble ESTs is by clustering. The goal of such a project is the construction of a gene index in which ESTs and full-length transcripts are partitioned into index classes (or clusters) such that they are placed in the same index class if and only if they represent the same gene. Projects related to EST clustering and assembly include UniGene from the National Center for Biotechnology Information; the TIGR Gene Index (http://www.tigr.org/tdb/hgi/hgi.html) from the Institute for Genomic Research; the Sequence Tag Alignment and Consensus Knowledgebase (STACK, http://ziggy.sanbi.ac.za/stack/stacksearch.htm); the Merck/Washington University Gene Index; and the GenExpress project. All of these projects perform some type of cluster analysis in which sequence similarity is used to form the clusters. For an overview of EST clustering, see, Win Hide and Alan Christoffels, EST Clustering Tutorial, ISMB, 1999 (available at www.sanbi.ac.za) and incorporated here by reference. It is worth noting that the gene indexing process typically incorporate information about EST and full length cDNA sequences.

Genomic Alignment and Assessment of Sequence Clusters

In one aspect of the invention, transcript sequences (such as EST sequences, full length cDNA sequences, consensus or examplar sequences from sequence clusters, etc.) are aligned to the genomic sequence to obtain information or verify information about the transcriptome. For example, in a preferred embodiment, human transcript sequences are aligned to the human genome sequence to verify the validity of the orientation s using consensus splice sites, detect chimeric UniGene clusters, determine dbEST genomic triming, etc. The alignment also provides information about transcribed locations in the genome.

Alignment of the transcript sequencies to the genomic sequence can be performed using for example, psLayout, a computer program available from University of California at Santa Cruz.

In an exemplary embodiment, genomic alignment starts with the following inputs: Genomic annotation branch annot.10 (genomic sequence and repeatMasker files for running pslayout); input data sets (mrna, cmrna, rsmrna, dbest, manual) used for psLayout against genome and Hs.data file for use in splitting PSL data into the cluster tree.

In this embodiment, PSL alignment files are generated (all, nbig, best) for alignments of the input sequences to the Golden Path genomic sequence data. The PSL data is then distributed into the cluster tree to the supercluster level for use by genomic assessment and later stages. There is no PSL header in this file. mkClusterTree is used to distribute the data to the cluster tree. For the WUSTL EST collection, a mock Hs.data file is created from a DBM hash of GI=>WUSTL location and the source Hs.data file (Hs.m4.data).

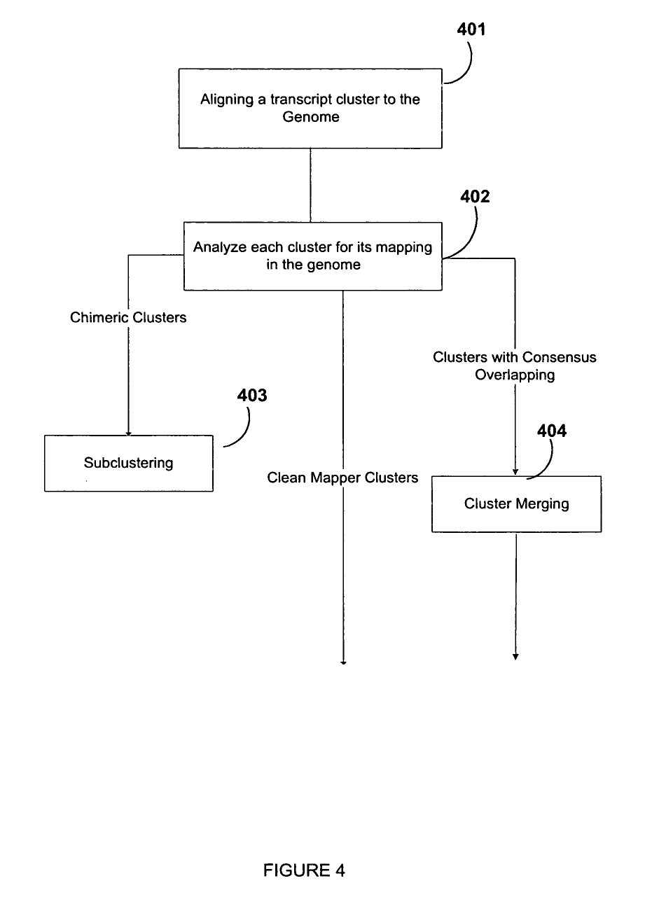

FIG. 4 shows several ways to use alignment of transcripts to the genome to annotate transcript sequence clusters such as EST clusters. Transcript sequences are aligned to the genome 401. The alignment of each sequence cluster is analyzed 402 to assess the cluster in view of the genomic sequence information. In preferred embodiments, several types of genomic assessment is performed:
1. Chimeric assessment:
A. Unaligned Cluster: Unaligned clusters are those for which none of the member sequences (and optionally the consensus sequence) align to the genome.
B. Cleanly Aligned Cluster: A cleanly aligned cluster is a cluster where the member sequences (and optionally the consensus) all align to the same genomic location. Unaligned sequence can be treated as belonging to this cluster, when all of the aligned sequences align to the same genomic location. Optionally, the unaligned sequences could disqualify a cluster as being cleanly aligned.
C. Chimeric Cluster: A chimeric cluster is a cluster where the member sequences (and optionally the consensus sequence) align to two or more positions. Unaligned sequence can be treated as either the same position where the majority of the sequences align or as a distinct “unaligned position”.
2. Assembly assessment: Assembly assessment is preferably performed by comparing the genomic assembly with the cluster assembly. Orientation and order of the member sequences is considered. If any of the sequences are not in the same orientation or order, then the cluster is flagged for further analysis.
3. Genomic annotation: At least two annotation alignments can be generated using the genomic position of the consensus. Any overlapping annotations (gene annotations and cDNA alignments) can be recorded as well as any splice consistent annotations. Alignments to the genome can be used to transfer annotations to the cluster. For instance, if a particlar disease loci is located in the same spot that the cluster is, then transfer that disease annotation to the cluster. Likewise, if an EST cluster that aligns to the same region as a gene prediction, the predicted gene annotations can be transferred to the cluster.
4. Cluster merging: Clusters with consensus and/or member sequences which overlap (e.g., within 1000 bases and optionally on the same strand) in genomic space, or clusters with consensus sequences and/or member sequences which are “evidence.” As used herein, “evidence” means they overlap in genomic space or that they share exonic sequence for the same genomic annotation (such as a predicted gene struction) are considered for merging.
5. External alignment assessment: Alignment information is collected from the external sources (for example, GenBank, RefSeq, dbEST, and UniGene). External alignment information is compared with sequence based in silico mapping. Clusters where the external alignment of the cluster itself or the member sequences disagree with our genomic alignments are annotated as such and potentially flagged for further analysis.

In addition to alignment of the transcript sequences to the genomic sequence, chromosome level alignment information can also be used for assessing transcript sequences, particularly transcript sequence clusters. Typically, chromosomal level alignment information is collected from GenBank, RefSeq, dbEST, and UniGene.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

1. A computer-implemented method for analyzing a plurality of transcript sequences in a cluster comprising:
aligning the transcript sequences in the cluster with their corresponding genomic sequences; and
determining the quality of the cluster according to the alignment; and
modifying the cluster according to the determined quality to improve alignment quality.

2. The method of claim 1 wherein the step of determining comprises further classifying a cluster as a chimeric cluster if the cluster is aligned to two separate locations in the genomic sequence.

3. The method of claim 2 wherein the chimeric cluster has at least 5% of its sequences aligned to each of the two separate locations.

4. The method of claim 3 wherein the chimeric cluster has at least 10% of its sequences aligned to each of the two separate locations.

5. The method of claim 4 wherein the chimeric cluster has at least 20% of its sequences aligned to each of the two separate locations.

6. The method of claim 5 wherein the chimeric cluster has at least 30% of its sequences aligned to each of the two separate locations.

7. The method of claims 3, 4, 5 or 6 further comprising subclustering the chimeric clusters; realigning subclusters' sequences to the genomic sequence; and analyzing the re-aligning to determine chimeric clusters.

8. The method of claim 7 wherein the process is repeated until no chimeric cluster is detected.

9. The method of claim 1 wherein the step of determining comprises detecting clusters with consensus sequences which overlap in genomic space.

10. The method of claim 9 further comprising merging the clusters with consensus sequences which overlap in genomic space.

11. The method of claim 1 wherein the step of determining comprises detecting clusters with consensus sequences within 1000 bases and on the same strand.

12. The method of claim 11 further comprising merging the clusters with consensus sequences within 1000 bases and on the same strand.

13. A method for triming a transcript sequence comprising: aligning the transcript sequence to its corresponding genomic sequence; removing a side sequence of the transcript sequence if the side is poorly aligned with the genomic sequence.

14. The method of claim 13 wherein the transcript sequence aligns with the genomic sequence with at least 80% identity.

15. The method of claim 14 wherein the transcript sequence aligns with the genomic sequence with at least 90% identity.

16. A computer-implemented method of designing a nucleic acid probe array comprising:
aligning a plurality of transcript sequences in a cluster to their corresponding genomic sequence;
modifying the clusters that are not optimally aligned to the genomic sequence to obtain at least one modified cluster wherein the modified cluster display an improved alignment to said genomic sequence; and
selecting probes targeting the at least one modified cluster to design the nucleic acid probe array.

17. The method of claim 16 wherein the step of modifying comprises subclustering chimeric clusters wherein a cluster is classified as a chimeric cluster if the cluster is aligned to two separate locations in the genomic sequence.

18. (canceled)

19. The method of claim 17 wherein the chimeric cluster has at least 5% of its sequences aligned to each of the two separate locations.

20. The method of claim 19 wherein the chimeric cluster has at least 10% of its sequences aligned to each of the two separate locations.

21. The method of claim 20 wherein the chimeric cluster has at least 20% of its sequences aligned to each of the two separate locations.

22. The method of claim 21 wherein the chimeric cluster has at least 30% of its sequences aligned to each of the two separate locations.

23. The method of claim 16 wherein the step of modifying comprises merging the clusters with consensus which overlap in genomic space.

24. The method of claims 16 further comprising merging the clusters with consensus within 1000 bases and on the same strand.

25. A method of designing a nucleic acid probe array comprising:
aligning a transcript sequence to its corresponding genomic sequence;
triming a side of the transcript sequence to obtain a trimmed transcript sequence if the side of the transcript sequence is poorly align with the genomic sequence; and
selecting probes targeting the trimmed transcript sequence or clusters including the trimmed transcript sequence.

26. A computer readable medium comprising computer-executable instructions for performing the method of analyzing a plurality of transcript sequences in a cluster comprising:
aligning transcript sequences from the cluster with genomic sequences;
determining the quality of the cluster according to the alignment; and
modifying the cluster according to the determined quality to improve alignment quality.

27. The computer readable medium of claim 26 wherein the step of determining further comprises classifying a cluster as a chimeric cluster if the cluster is aligned to two separate locations in the genomic sequence.

28. The computer readable medium of claim 27 wherein the chimeric cluster has at least 5% of its sequences aligned to each of the two separate locations.

29. The computer readable medium of claim 28 wherein the chimeric cluster has at least 10% of its sequences aligned to each of the two separate locations.

30. The computer readable medium of claim 29 wherein the chimeric cluster has at least 20% of its sequences aligned to each of the two separate locations.

31. The computer readable medium of claim 30 wherein the chimeric cluster has at least 30% of its sequences aligned to each of the two separate locations.

32. The computer readable medium of claims 28, 29, 30 or 31 further comprising subclustering the chimeric clusters; realigning subclusters' sequences to the genomic sequence; and analyzing the re-aligning to determine chimeric clusters.

33. The computer readable medium of claim 32 wherein the process is repeated until no chimeric cluster is detected.

34. The computer readable medium of claim 33 wherein the step of determining comprises detecting clusters with a consensus sequence that overlaps in the genomic space.

35. The computer readable medium of claim 34 further comprising merging the clusters with consensus sequence which overlap in genomic space.

36. The computer readable medium of claim 35 wherein the step of determining comprises detecting clusters with consensus sequences within 1000 bases and on the same strand.

37. The computer readable medium of claim 36 further comprising merging the clusters with consensus sequences within 1000 bases and on the same strand.

38. A computer readable medium comprising computer-executable instructions for performing the method comprising: aligning a transcript sequence to its corresponding genomic sequence; removing a side sequence of the transcript sequence if the side is poorly aligned with the genomic sequence.

39. The computer readable medium of claim 38 wherein the transcript sequence aligns with the genomic sequence with at least 80% identity.

40. The computer readable medium of claim 39 wherein the transcript sequence aligns with the genomic sequence with at least 90% identity.

41. A computer readable medium comprising computer-executable instructions for performing the method of designing a nucleic acid probe array comprising:
aligning a plurality of transcript sequences in a cluster to their corresponding genomic sequence;
modifying the cluster that are not optimally aligned to the genomic sequence to obtain at least one modified cluster wherein the modified cluster display an improved alignment to said genomic sequence; and
selecting probes targeting the at least one modified cluster to design the nucleic acid probe array.

42. The computer readable medium of claim 41 wherein the step of modifying comprises subclustering a chimeric cluster wherein a cluster is classified as a chimeric cluster if the cluster is aligned to two separate locations in the genomic sequence.

43. (canceled)

44. The computer readable medium of claim 42 wherein the chimeric cluster has at least 5% of its sequences aligned to each of the two separate locations.

45. The computer readable medium of claim 44 wherein the chimeric cluster has at least 10% of its sequences aligned to each of the two separate locations.

46. The computer readable medium of claim 45 wherein the chimeric cluster has at least 20% of its sequences aligned to each of the two separate locations.

47. The computer readable medium of claim 46 wherein the chimeric cluster has at least 30% of its sequences aligned to each of the two separate locations.

48. The computer readable medium of claim 47 wherein the step of modifying comprises merging the clusters with consensus which overlap in genomic space.

49. The computer readable medium of claims 48 further comprising merging the clusters with consensus sequences within 1000 bases and on the same strand.

50. A computer readable medium comprising computer-executable instructions for performing the method of
aligning a transcript sequence to its corresponding genomic sequence;
triming a side of the transcript sequence to obtain a trimmed transcript sequence if the side of the transcript sequence is poorly align with the genomic sequence; and
selecting probes targeting the trimmed transcript sequence or clusters including the trimmed transcript sequence.
